(19) United States
(12) Patent Application Publication
(10) Pub. No.: US 2005/0277905 A1
(43) Pub. Date: Dec. 15, 2005

(54) Diapers, diaper fasteners, and/or diaper landing areas

(76) Inventors: William G. Pedersen, North Canton, OH (US); Donald Vincent Varanese, Mentor, OH (US); Gary D. Waechter, Cincinnati, OH (US)

Correspondence Address: RENNER, OTTO, BOISSELLE & SKLAP, LLP, 1621 EUCLID AVE, 19TH FL, CLEVELAND, OH, 44115-2191, US

(21) Appl. No.: 11/148,723

(22) Filed: Jun. 9, 2005

Related U.S. Application Data
(60) Provisional application No. 60/578,441, filed on Jun. 9, 2004.

(51) Int. Cl. A61F 13/15

(52) U.S. Cl. 604/389

(57) ABSTRACT

Diapers, diaper fasteners, and diaper landing areas which incorporate cohesively engaging or engagable surfaces to provide selective closure and/or selective removal of a diaper from a user. The cohesive surfaces can be printed, coated, sprayed, extruded on, co-extruded with, or otherwise applied to the host substrate. Alternatively, the cohesive surfaces can be integral with the host substrate or material. In either case, the cohesive engagement preferably occurs upon contact and slight (e.g., manually applied pressure) pressure and/or preferably occurs at room temperature (e.g., heat is not required).

### RELATED APPLICATION

This application claims priority under 35 U.S.C. §119(e) to U.S. Provisional Patent Application No. 60/578,441 filed on Jun. 9, 2004. The entire disclosure of this provisional application is hereby incorporated by reference.

### FIELD OF THE INVENTION

This invention relates generally, as indicated, to diapers, diaper fastening tabs, and/or diaper landing zones.

### BACKGROUND OF THE INVENTION

A disposable diaper usually comprises a laminate having a liquid absorbent pad enclosed within a liquid permeable inner shell and a liquid impermeable outer shell. Although diapers are often associated with babies and young children, these type of garments are also widely used by adults with incontinence or other issues. In any event, fasteners, which engage in appropriately placed landing areas on the diaper, can be used to provide closure about the wearer. Alternatively, the diapers can be of a “pull-up” style, with selectively separable seams to remove a soiled diaper from a wearer.

### SUMMARY OF THE INVENTION

The present invention provides diapers, diaper fasteners, and diaper landing areas which incorporate cohesively engaging or engagable surfaces to provide selective closure and/or selective removal of a diaper from a user.

### DRAWINGS

FIG. 1A is a perspective view of a disposable diaper with a pair of fastening tabs.

FIG. 1B is a side view of a diaper fastening tab in a pre-installation condition, the tab including a cohesively engaging surfaces on its landing section and its fastening section.

FIG. 1C is a perspective view of a roll which can be laterally cut at desired intervals to provide a plurality of the fastening tabs.

FIG. 1D is a side view of the fastening tab upon partial installation onto the diaper.

FIG. 1E is a side view of the fastening tab upon complete installation onto the diaper.

FIG. 1F is a side view of the fastening tab upon initially securing the diaper onto the wearer.

FIG. 1G is a side view of the fastening tab as it is being opened.

FIG. 1H is a view similar to that of FIG. 1B, except that the fastening tab includes a modified fastening section.

FIG. 2A is an elevation view of a fastening tab having a cohesively engagable surface on its fastening section, the tab being shown in a folded storage condition.

FIG. 2B is a perspective view of a tab in a deployed condition.

FIG. 2C is a view similar to FIG. 2A, except that the fastening tab has a modified adhesive layer.

FIG. 2D is a view similar to FIG. 2A, except that the fastening tab has a modified adhesive layer.

FIGS. 3A-3E are perspective views of other diaper fastening tabs with cohesively engagable surfaces on their fastening sections.

FIGS. 4A and 4B are top views of a diaper which includes cohesive surfaces, the diaper being shown in an open position and closed position, respectively.

FIGS. 5A and 5B are top plan views of a diaper having cohesive surfaces on integral portions thereof.

FIG. 6 a side view of a diaper having cohesive surfaces forming releasable side seams.

### DETAILED DESCRIPTION

The present invention provides diapers, diaper fasteners, and diaper landing zones which incorporate cohesive layers, cohesive films, cohesive coatings, and/or other forms of cohesive materials to provide cohesively-engaging surfaces for selectively closing and/or removing a diaper from a wearer. “Cohesively-engaging” in the context of the present invention refers to a property between surfaces which enables them to be adhered aggressively together, while not adhering to other surrounding (and dissimilar) surfaces. The cohesive engagement preferably occurs upon contact and slight (e.g., manually applied pressure) pressure and/or preferably occurs at room temperature (e.g., heat is not required).

The cohesive materials can be prepared from polymers which have cohesive properties. Cohesive property is the property of adhering to material of like nature and is known to those in the art. (See e.g., U.S. Pat. No. 5,085,655 which is assigned to the assignee of the present invention and the entire disclosure of which is hereby incorporated by reference.)

The cohesive is typically nonadhesive to dissimilar materials. These polymers are know as cohesive or autoadhesive polymers. The cohesive layers are typically a thermoplastic elastomer material having cohesive properties at room temperature. The cohesive materials are characterized by physical cross-links which are labile and therefore may be rendered ineffective by processing techniques involving the application of heat.

Cohesive materials include multiblock copolymers of radial, triblock and diblock structures including non-rubbery segments of mono- and polycyclic aromatic hydrocarbons, and more particularly, mono- and polycyclic arenes. Illustrative mono- and polycyclic arenes include substituted and unsubstituted poly(vinyl)arenes of monocyclic and bicyclic structure. The cohesive materials can include non-rubbery segments of substituted or unsubstituted monocyclic arenes of sufficient segment molecular weight to assure phase separation at room temperature.

The cohesive materials may be natural rubbers, or may be synthetic rubbers (e.g., styrenic rubbers, ethylene propylene rubbers, and mixtures thereof). The cohesive materials can comprise at least one rubber based elastomer materials. The rubber elastomer comprises linear, branched, or radial block copolymers represented by the diblock structure A-B, the triblock A-B-A, the radial or coupled structures (A-B)ₙ, and combinations of these where A represents a hard thermoplastic phase or block which is non-rubbery or glassy or crystalline at room temperature but fluid at higher temperatures, and B represents a soft block which is rubbery or elastomeric at service or room temperature. The cohesive materials may comprise from about 75% to about 95% by weight of rubbery segments and from about 5% to about 25% by weight of non-rubbery segments.

The non-rubbery segments or hard blocks comprise polymers of mono- and polycyclic aromatic hydrocarbons, and more particularly vinyl-substituted aromatic hydrocarbons which may be monocyclic or bicyclic in nature. Useful rubbery blocks or segments are polymer blocks of homopolymers or copolymers of aliphatic conjugated dienes. Rubbery materials such as polyisoprene, polybutadiene, and styrene butadiene rubbers may be used to form the rubbery block or segment. Particularly useful rubbery segments include polydienes and saturated olefin rubbers of ethylene/butylene or ethylene/propylene copolymers. The latter rubbers may be obtained from the corresponding unsaturated polyalkylene moieties such as polybutadiene and polyisoprene by hydrogenation thereof.

The block copolymers of vinyl aromatic hydrocarbons and conjugated dienes which may be utilized include any of those which exhibit elastomeric properties. The block copolymers may be diblock, triblock, multiblock, starblock, polyblock or combinations thereof. Throughout this specification and claims, the terms diblock, triblock, starblock, multiblock, and polyblock with respect to the structural features of block copolymers are to be given their normal meaning as defined in the literature such as in the Encyclopedia of Polymer Science and Engineering, Vol. 2, (1985) John Wiley & Sons, Inc., New York, pp. 325-326, and by J. E. McGrath in Block Copolymers, Science Technology, Dale J. Meier, Ed., Harwood Academic Publishers, 1979, at pages 1-5.

Such block copolymers may contain various ratios of conjugated dienes to vinyl aromatic hydrocarbons including those containing up to about 40% by weight of vinyl aromatic hydrocarbon. Accordingly, multi-block copolymers may be utilized which are linear or radial symmetric or asymmetric and which have structures represented by the formulae A-B, A-B-A, A-B-A-B, B-A-B, (AB)_{0,1,2} . . . BA, etc., wherein A is a polymer block of a vinyl aromatic hydrocarbon or a conjugated diene/vinyl aromatic hydrocarbon tapered copolymer block, and B is a rubbery polymer block of a conjugated diene.

The block copolymers may be prepared by any of the well-known block polymerization or copolymerization procedures including sequential addition of monomer, incremental addition of monomer, or coupling techniques as illustrated in, for example, U.S. Pat. Nos. 3,251,905; 3,390,207; 3,598,887; and 4,219,627. As well known, tapered copolymer blocks can be incorporated in the multi-block copolymers by copolymerizing a mixture of conjugated diene and vinyl aromatic hydrocarbon monomers utilizing the difference in their copolymerization reactivity rates. Various patents describe the preparation of multi-block copolymers containing tapered copolymer blocks including U.S. Pat. Nos. 3,251,905; 3,639,521; and 4,208,356, the disclosures of which are hereby incorporated by reference.

Conjugated dienes which may be utilized to prepare the polymers and copolymers are those containing from 4 to about 10 carbon atoms and more generally, from 4 to 6 carbon atoms. Examples include from 1,3-butadiene, 2-methyl-1,3-butadiene(isoprene), 2,3-dimethyl-1,3-butadiene, chloroprene, 1,3-pentadiene, 1,3-hexadiene, etc. Mixtures of these conjugated dienes also may be used. The preferred conjugated dienes are isoprene and 1,3-butadiene. Examples of vinyl aromatic hydrocarbons which may be utilized to prepare the copolymers include styrene and the various substituted styrenes such as o-methylstyrene, p-methylstyrene, p-tert-butylstyrene, 1,3-dimethylstyrene, alpha-methylstyrene, beta-methylstyrene, p-isopropylstyrene, 2,3-dimethylstyrene, o-chlorostyrene, p-chlorostyrene, o-bromostyrene, 2-chloro-4-methylstyrene, etc. The preferred vinyl aromatic hydrocarbon is styrene.

Many of the above-described copolymers of conjugated dienes and vinyl aromatic compounds are commercially available. The number average molecular weight of the block copolymers, prior to hydrogenation, is from about 20,000 to about 500,000, or from about 40,000 to about 300,000. Here and elsewhere in the specification and claims, the range and ratio limits may be combined. The average molecular weights of the individual blocks within the copolymers may vary within certain limits. In most instances, the vinyl aromatic block will have a number average molecular weight in the order of about 2000 to about 125,000, and or between about 4000 and 60,000. The conjugated diene blocks either before or after hydrogenation will have number average molecular weights in the order of about 10,000 to about 450,000 and or from about 35,000 to 150,000.

Also, prior to hydrogenation, the vinyl content of the conjugated diene portion generally is from about 10% to about 80%, and the vinyl content is typically from about 25% to about 65%, or from about 35% to about 55% when it is desired that the modified block copolymer exhibit rubbery elasticity. The vinyl content of the block copolymer can be measured by means of nuclear magnetic resonance. Specific examples of diblock copolymers include styrene-butadiene (SB), styrene-isoprene (SI), and the hydrogenated derivatives thereof. Examples of triblock polymers include styrene-butadiene-styrene (SBS), styrene-isoprene-styrene (SIS), alpha-methylstyrene-butadiene-alpha-methylstyrene, and alpha-methylstyrene-isoprene alpha-methylstyrene. Examples of commercially available block copolymers useful as the adhesives in the present invention include those available from Shell Chemical Company and include Styrene/Rubber Kraton D1101 (Linear SBS), D1107P (Linear SIS), D1111 (Linear SIS), D1112P (Linear SIS), D1113P (Linear SIS), D1117P (Linear SIS), and D1320X (Multi-arm (Sl)n).

Upon hydrogenation of the SBS copolymers comprising a rubbery segment of a mixture of 1,4 and 1,2 isomers, a styrene-ethylene-butylene styrene (SEBS) block copolymer is obtained. Similarly, hydrogenation of an SIS polymer yields a styrene-ethylene propylene-styrene (SEPS) block copolymer.

The selective hydrogenation of the block copolymers may be carried out by a variety of well known processes including hydrogenation in the presence of such catalysts as Raney nickel, noble metals such as platinum, palladium, etc., and soluble transition metal catalysts. Suitable hydrogenation processes which can be used are those wherein the diene-containing polymer or copolymer is dissolved in an inert hydrocarbon diluent such as cyclohexane and hydrogenated by reaction with hydrogen in the presence of a soluble hydrogenation catalyst. Such procedures are described in U.S. Pat. Nos. 3,113,986 and 4,226,952, the disclosures of which are incorporated herein by reference. Such hydrogenation of the block copolymers which are carried out in a manner and to extent as to produce selectively hydrogenated copolymers having a residual unsaturation content in the polydiene block of from about 0.5% to about 20% of their original unsaturation content prior to hydrogenation.

In one embodiment, the conjugated diene portion of the block copolymer is at least 90% saturated and more often at least 95% saturated while the vinyl aromatic portion is not significantly hydrogenated. Particularly useful hydrogenated block copolymers are hydrogenated products of the block copolymers of styrene—isoprene-styrene such as a styrene-(ethylene/propylene)-styrene block polymer. When a polystyrene-polybutadiene-polystyrene block copolymer is hydrogenated, it is desirable that the 1,2-polybutadiene to 1,4-polybutadiene ratio in the polymer is from about 30:70 to about 70:30. When such a block copolymer is hydrogenated, the resulting product resembles a regular copolymer block of ethylene and 1-butene (EB). As noted above, when the conjugated diene employed as isoprene, the resulting hydrogenated product resembles a regular copolymer block of ethylene and propylene (EP).

In one embodiment, the cohesive material comprises SEBS block copolymers sold by the Shell Chemical Company under the designations KRATON G1650, G1652 and G1657. KRATON G1650 and G1652 are primarily of triblock structure and each has a styrene/rubber ratio of about 30/70. KRATON G1657 is a mixture of triblock and diblock structures in about a 70/30 ratio and has a styrene/rubber ratio of about 13/87.

In one embodiment, the cohesive material is an ethylene propylene rubber or EP rubber. The materials are also known as EPM and EPDM rubbers. These materials are known to those in the art. The ethylene-propylene rubbers include ethylene propylene copolymers including random copolymers, and terpolymers of ethylene and propylene with nonconjugated dienes, such as those described above, and particularly 5-ethyldiene-2-norbornene, 1,8 octadiene, 1,4 hexadiene cyclopentadiene (EPDM) and the like. EP rubbers are commercially available from a number of sources. One example is EPDM rubbers available from Exxonmobil under the tradename Vistalon.

In one embodiment, the cohesive materials contains less than 5%, or less than 1% by weight tackifier. In another embodiment, the cohesive material is free of tackifier. In one embodiment, the cohesive material contains less than 5%, or less than 1% by weight plasticizer. In another embodiment, the cohesive material is free of plasticizer. In another embodiment, the cohesive materials contain less than 5%, or less than 1% by weight of polyurethane. In another embodiment, the cohesive materials are free of polyurethane. In another embodiment, the composition contains less than 5%, or less than 1% by weight of polymers of styrene and acrylic or methacrylic acids or esters. In one embodiment, the cohesive material is free of polymers of styrene and acrylic or methacrylic acids or esters. In another embodiment, the cohesive materials contain less than 5% by weight, or less than 1% by weight of a polymer derived from vinyl pyrrolidone. These polymers include homopolymers as well as copolymers. In one embodiment, the cohesive material is free of any polymers derived from vinyl pyrrolidone.

The cohesive films, layers or coatings can have a thickness of about 0.5 to about 200, or about 2 to about 100, or about 4 to about 50 microns. The thickness of cohesively engaging films/layers/coatings can be same, or can be different, depending upon intended use, manufacturing methods, and/or other factors.

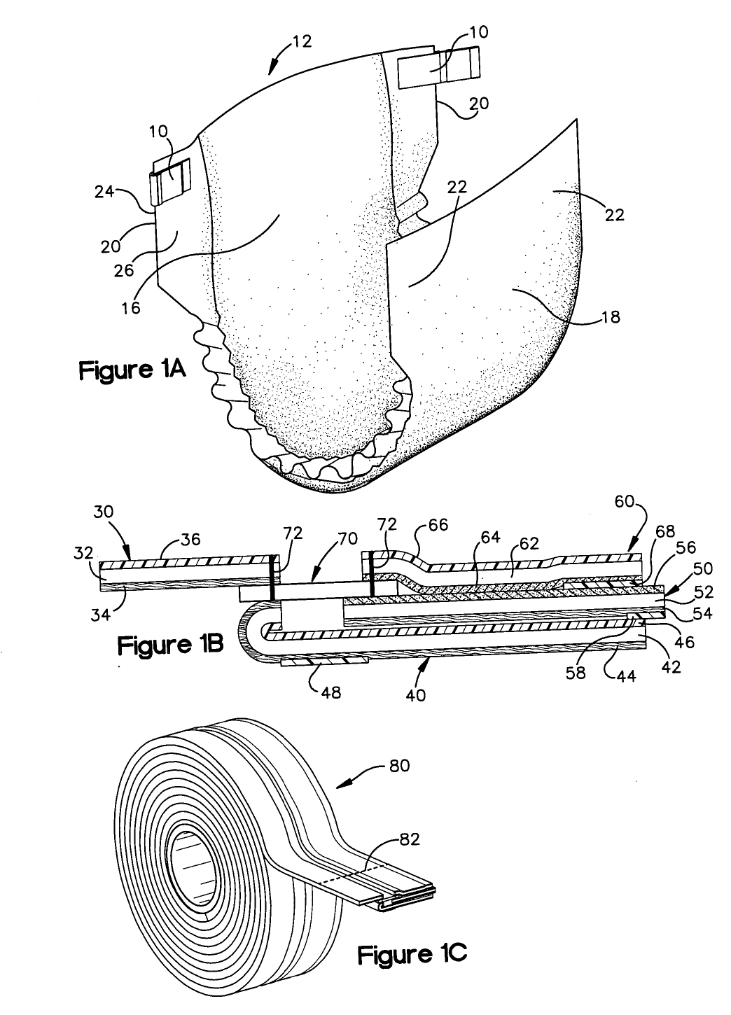

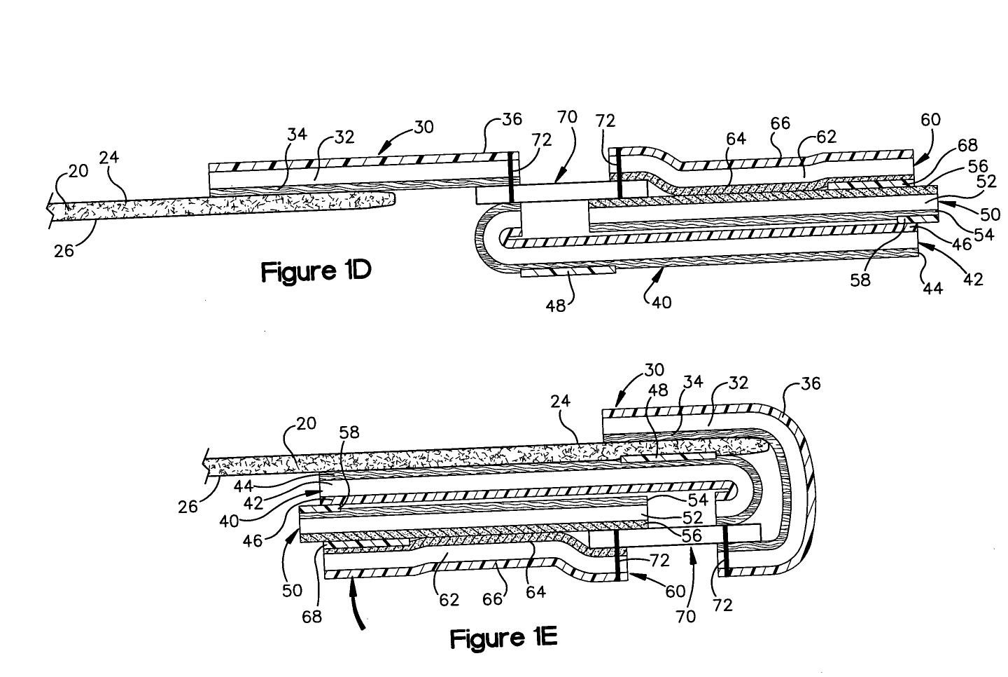

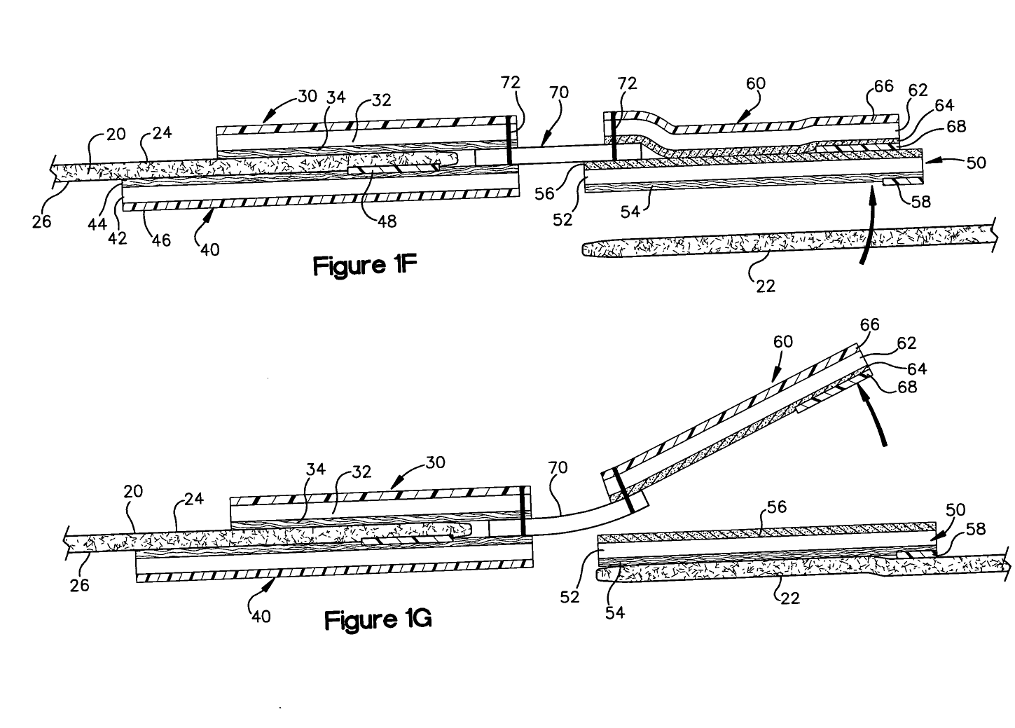

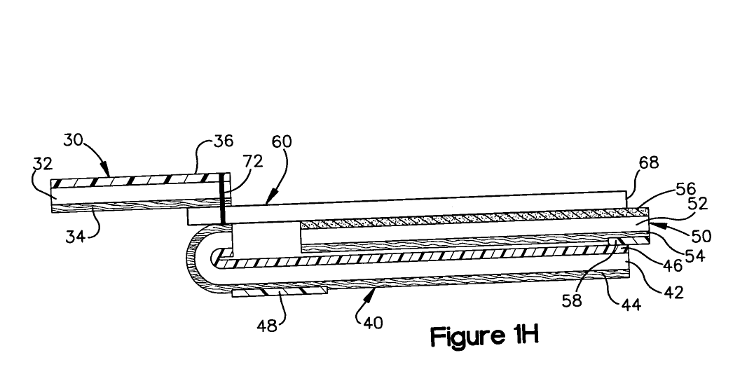

Referring now to FIGS. 1A-1H, and initially to FIG. 1A, a pair of fasteners 10 according to the present invention are shown installed on a disposable diaper 12. The left hand fastener 10 is shown in a storage condition and the right hand fastener 10 is shown in a deployed condition. The illustrated disposable diaper 12 comprises a laminate having a liquid absorbent pad enclosed within a liquid permeable inner shell and a liquid impermeable outer shell. The diaper 12 has a rear portion 16 which is intended to cover the wearer's behind and a front portion 18 which is intended to cover the wearer's front. The fasteners 10 are respectively attached to edges 20 of the rear portion 16 and are arranged to provide closure about the wearer upon engagement with respective landing areas 22 on the front portion 18 of the diaper. The edges 20 can be viewed as having an outer surface 24 and an inner surface 26.

In FIG. 1B, a fastener 10 is shown in an installation condition. The fastener 10 comprises a first attachment section 30, a second attachment section 40, a landing section 50, a fastening section 60, and a stretchable section 70. The first attachment section 30 comprises a substrate 32 having an adhesive 34 on one surface and a release coating 36 on the opposite surface. The second attachment section 40 comprises a substrate 42 having an adhesive 44 on one surface and a release coating 46 on an opposite surface. A stick-protection pad 48 is provided on the lower surface of the substrate 42 and is aligned with the stretchable section 70. The landing section 50 comprises a substrate 52 having an adhesive 54 on one surface and a cohesive layer 56 on the opposite surface. The fastening section 60 comprises a substrate 62 having a cohesive layer 64 on one surface and a release coating 66 on the opposite surface. Finger tabs 58 and 68 are provided on the landing section 50 and the fastening section 60, respectively. The stretchable section 70 can comprise a piece of elastic material and seams 72 are used to connect the stretchable section 70 to the appropriate other sections of the fastener 10.

As is best seen by referring briefly to FIG. 1C, the fasteners 10 in the pre-installation condition shown in FIG. 1B can be cut from a stock roll 80, preferably in a high-speed efficient manufacturing manner.

To secure the fastener 10 to the diaper 12, the attachment section 30 is permanently secured to the outer surface 24 of the diaper's edge 20 by the adhesive 34. (FIG. 1D.) Then, the attachment section 40, the landing section 50, the fastening section 60, and the stretch section 70 are folded over, and the attachment section 40 is permanently secured to the inner surface 26 via the adhesive 44. (FIG. 1E). To fit the diaper 12 onto a wearer, the fastener 10 is first placed into a deployed condition by grasping the finger tab 58 and unfolding the landing section 50 (and the fastening section 60 carried therewith) away from the attachment section 40. The front portion 18 of the diaper 12 can then be fitted over the wearer and the landing section 50 permanently secured to the landing area 22 by the adhesive 54. (FIG. 1F.) To open the fastener 10, the opening finger tab 68 is grasped and the fastening section 60 is folded away from the now-permanently-secured-to-the-diaper landing section 50. (FIG. 1G.) Assuming the cohesive 56 and/or the cohesive 64 is of an appropriate type, the fastener 10 can be subsequently re-closed by repositioning the fastening section 60 onto the landing section 50.

As shown in FIG. 1H, the fastening section 60 and the elastic section 70 can be replaced by a single section 60 of elastic material with the cohesive 64 coated (or otherwise carried) by a distal end of the section 60.

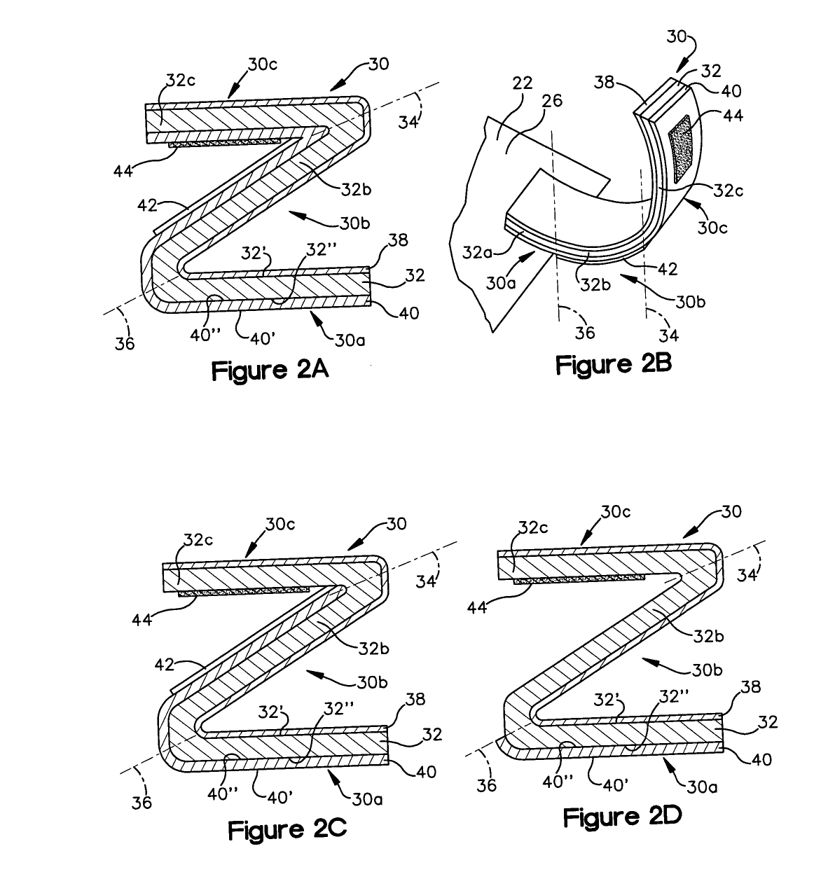

Referring now to FIGS. 2A-2D, and initially to FIG. 2A, a diaper tab 30 is shown which includes a substrate 32 having intermediate fold lines or planes 34 and 36. The fold lines 34 and 36 divide the tab 30 and substrate 32 into an attachment section 30a/32a, a central section 30b/32b, and a fastening section 30c/32c. A release coating layer 38 extends along the substrate's outer surface 32′, at least over the attachment section 32a. An adhesive layer 40 (with an outer surface 40′ and an inner surface 40″) extends along the substrate's inner surface 32″ at least over the attachment section 32a. A release coating 42 can extend along the outer surface 40′ of the adhesive layer 40 across the central section 30b.

As is best seen by referring briefly to FIG. 2B, the attachment section 30a/32a is mounted to the longitudinal end 26 of a diaper 22 by the adhesive layer 40.

A cohesive layer 44 forms the innermost surface of the fastening section 30c/32c. As shown in FIG. 2A, the adhesive layer 40 can extend along the entire surface along the substrate's inner surface 32″ and the cohesive layer 44 can be bonded to the adhesive layer 40 in the fastening section 30c/32c. Alternatively, the adhesive layer 40 can extend along only the attachment section 30a/32a and the central section 30b/32b (FIG. 2C) or only along the attachment section 30a/32a (FIG. 2D). In any event, the diaper 22 (FIG. 2B) would also include a landing area which cohesively joins with the cohesive layer 44 to close the diaper.

As was indicated above, the release coating 38 extends at least over the attachment section 32a. The release coating 38 is provided to prevent “sticking” of the inner surface 40″ of the adhesive layer 40 to the outer surface 32′ of the substrate 32, and thus only needs to be positioned in those areas where sticking, due to the adhesive layer 40 or cohesive layer 44, will be a problem. For example, the adhesive layer 40 will not present sticking issues in the fastening section 32c in FIGS. 2A, 2C and 2D, and will not present sticking issues in the central section 32a in FIG. 2D. As for the release coating 38 in the fastening section 32c, it may or may not be necessary depending upon the substrate material and the make-up of the cohesive in the cohesive layer 44. That being said, from a manufacturing point of view, it might be easier to just coat the substrate's entire outer surface 32′, regardless of the extent of the adhesive layer 40 and/or the sticking susceptibly of the cohesive layer 44.

As was also indicated above, the release coating 42 can extend across the central section 30b. The release coating 42 is provided to prevent “sticking” of the central section 30b/32c to the cohesion layer 44 during deployment of the tab 30. Thus, depending upon the substrate material and/or the cohesive chemistry, such a release coating may or may not be necessary.

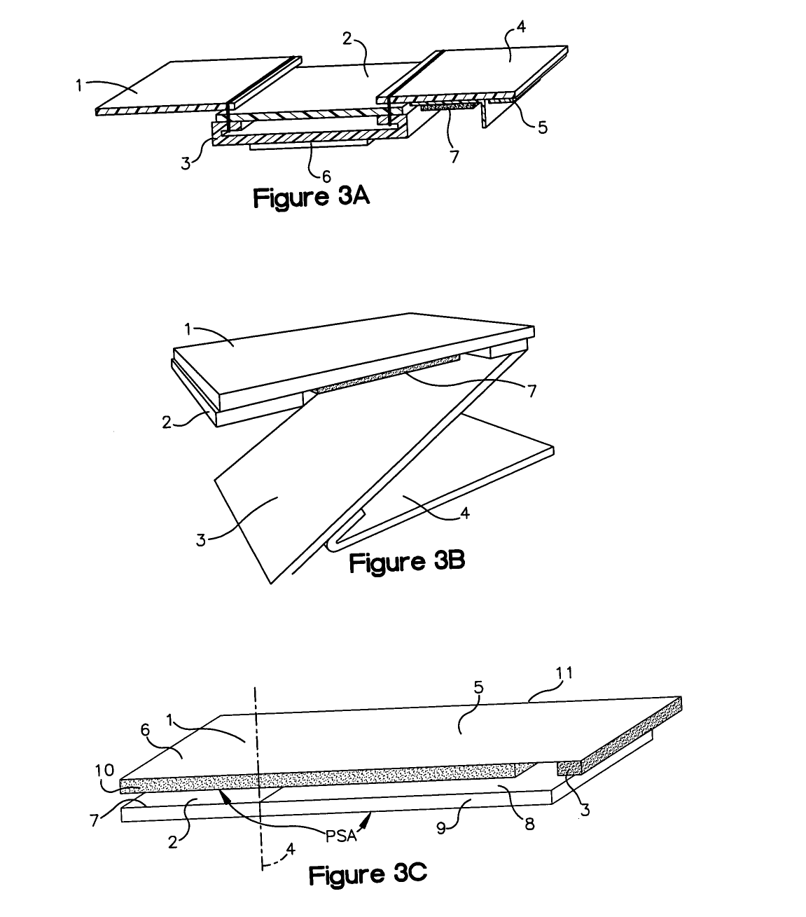

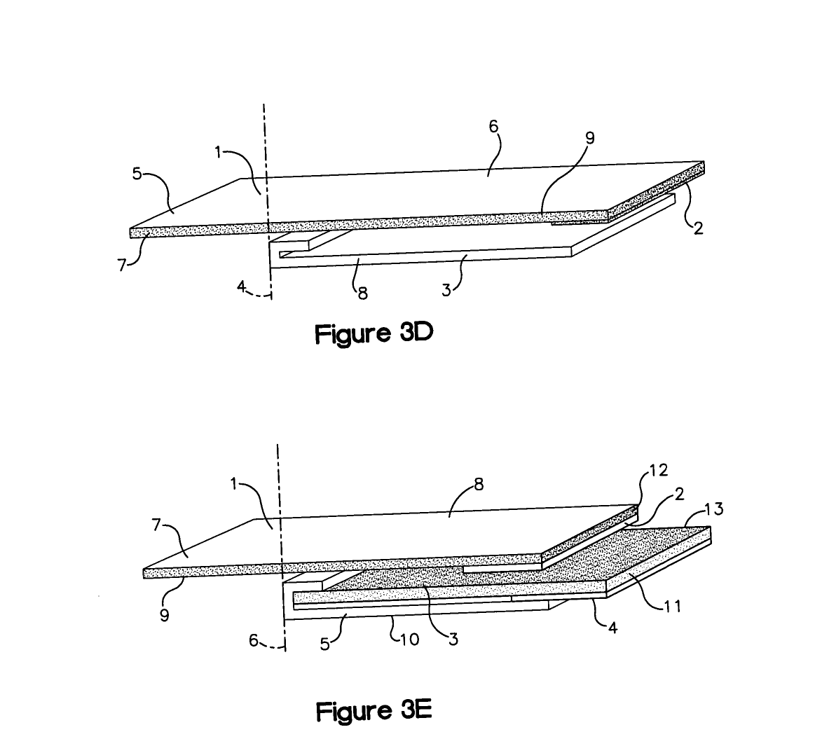

Referring now to FIGS. 3A-3E, other forms of diaper fastening tabs are shown. In FIG. 3A, the tab includes a first attachment section 1, an elastic section 2, a second attachment section 3, a fastening section 4, a fingerlift 5 and a cover stip 6. The first attachment section 1 and the fastening section are elastically joined by the section 2. A cohesive surface 7 is provided on the fastening section 4 for cohesive engagement with the landing area of the diaper.

In FIG. 3B, the tab includes a fastening section 1, a fingerlift 2, a central section 3, and an attachment section 4. The attachment section, the central section, and the fastening section are folded into a Z-shape when the tab is in a storage condition. The sections are made of separate portions of a substrate (or substrates) and are connected together by Y-bonds. A cohesive surface 7 is provided on the fastening section 1 for cohesive engagement with the landing area of the diaper.

In FIG. 3C, the tab includes a first strip 1, a second strip 2, and a fingerlift 3. A fold line 4 divides the first strip 1 into a fastening section 5 and an overlay section 6, and divides the second strip 2 into a first attachment section 7 and a second attachment section 8. An adhesive 9 on the second strip 2 secures the tab to the anchoring area of the diaper. An adhesive 10 on the overlay section 6 secures this section to the first attachment section 7. A cohesive surface 11 is provided on the fastening section 5 for cohesive engagement with the landing area of the diaper.

In FIG. 3D, the tab includes a first strip 1, a fingerlift 2, and a second strip 3. A fold line 4 divides the first strip 1 into an attachment section 5 and a fastening section 6. An adhesive 7 on the attachment section 5 secures the tab to the anchoring area of the diaper. An adhesive 8 on the second strip 3 also secures the tab to the anchoring area of the diaper and additionally connects the second strip 3 to the first strip 1. A cohesive surface 9 is provided on the fastening section 6 for cohesive engagement with the landing area of the diaper.

In FIG. 3E, the fastening tab includes a first strip 1, a fingerlift 2, a landing section 3, and a fingerlift 4, and a second strip 5. A fold line 6 divides the first strip 1 into an attachment section 7 and a fastening section 8. Adhesive 9 on the attachment section 7 secures the tab to the anchoring area of the diaper, adhesive 10 on the strip 5 also secures the tab to the anchoring area of the diaper, and adhesive 11 on the landing section 3 secures this section to the landing area of the diaper. A cohesive surface 12 and a cohesive surface 13 are provided on the fastening section 8 and the landing section 3.

The substrates or facestocks can be made of cloth, kraft paper, cellophane film, non-woven webs, polymeric films (e.g., polypropylene, polyvinyl chloride, polyethylene terephthalate, and polyethylene) or other suitable materials or laminates. The adhesives can be any conventional adhesive, including pressure sensitive adhesives and non-pressure sensitive adhesives. Suitable pressure sensitive adhesives include acrylic resin and natural or synthetic based rubber adhesives, such as those set forth in U.S. Pat. No. 3,932,328 (the entire disclosure of which is hereby incorporated by reference). The release coatings can be a silicone coating, a carbamate coating, or any other coating which promotes deployment of the relevant adjacent section.

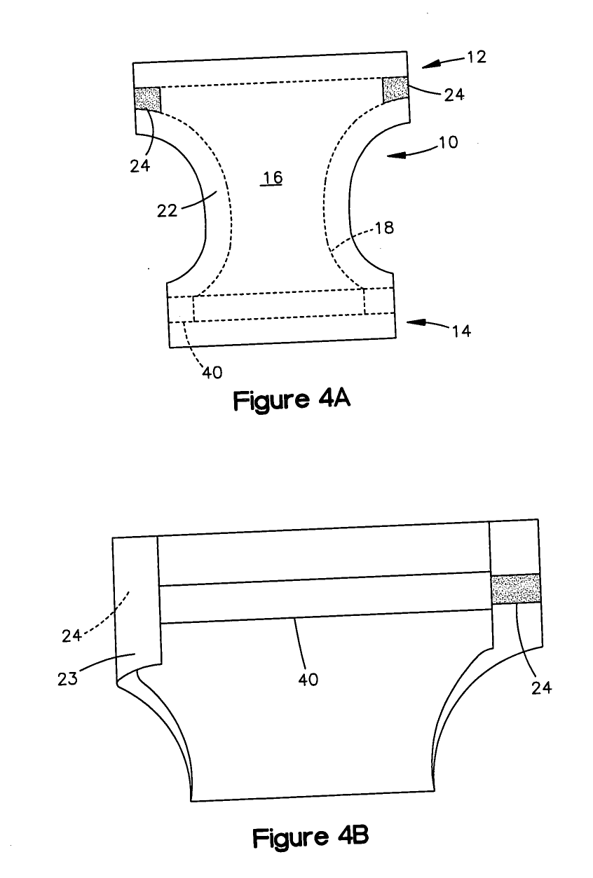

The diaper fasteners can also be of a “tabless” design as is shown in FIGS. 4A and 4B. In this design, the diaper 10 has first and second ends 12 and 14, a moisture-pervious inner layer 16, an absorbent pad batt 18, and a moisture-impervious outer layer 20. The inner and outer layers 16 and 20 are secured to one another laterally outwardly of the absorbent batt 18 to provide at the periphery of the configuration a flexible two-layer battless diaper margin 22. A cohesive surface 24 is provided on each lateral side of the first end 12 of the diaper. A cohesive surface 40 is provided on the diaper's outer layer 20 near the second or front diaper end 14, and positioned to receive the cohesive surface 24 when the diaper is closed, as shown in FIG. 4B.

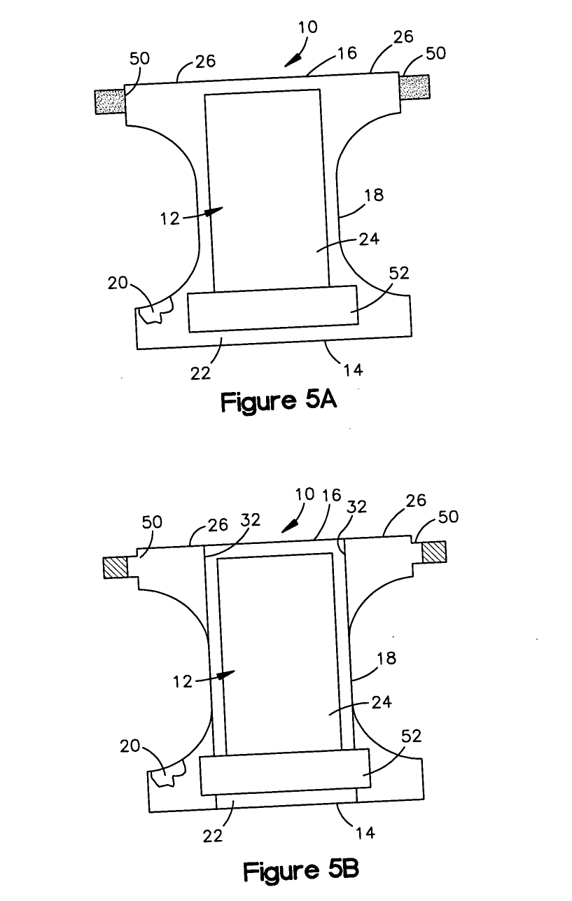

The diaper fasteners need not be separately formed from the diaper itself. As is shown in FIG. 5A, the diaper 10 is formed solely from a chassis 12 having a front portion 14, a rear portion 16, and a crotch portion 18 therebetween. An absorbent core 24 is interposed within the topsheet 20 and the backsheet 22 of the chassis 12. The rear portion 16 includes a pair of integral ears 26 (i.e., the ears are formed in one piece with the rest of the rear portion 16) and the ears 26 each include an integral extensions or strips 50 (i.e., each strip is formed in one piece with rest of the ear). A cohesive landing area 52 (i.e., an area having a cohesive surface) is located on the backsheet 22 and engages with cohesive surfaces on the strips 50 to close the diaper.

Alternatively, as shown in FIG. 5B, the diaper can be formed from a rectangular chassis 12 with separate ears attached thereto at a seam 32 to provide the diaper with an overall hourglass shape. In this case, the ears 26 would each still include an integral extension or strip 50 (i.e., a strip is formed in one piece with rest of the ear 26) with a cohesive surface thereon. The individual ear pieces 26 may be bonded to the chassis 12 using any suitable method, such as adhesive bonding, cohesive bonding, ultrasonic bonding, heat bonding, pressure bonding, friction bonding, autogenous bonding or combinations of bonding methods. It may be noted that if cohesive bonding is used to join the ears 26 to the chassis 12, this bonding cohesive could be applied at the same time as the fastening cohesive surfaces during the manufacturing process.

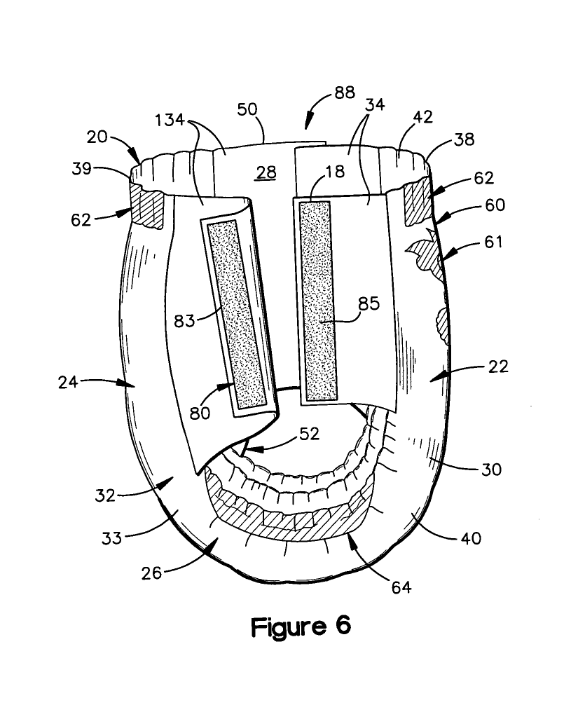

Referring now to FIG. 6, the diaper can instead be of a “pull-up” type with the cohesive surfaces forming “breakable” seams for sanitary removal of a soiled unit. The illustrated diaper 20 comprises an absorbent chassis 32 (defining a front waist region 22, a back waist region 24, a crotch region 26, an inner surface 28, and an outer surface 30) which comprises a rectangular structure 33, a pair of transversely opposed front side panels 34, and a pair of transversely opposed back side panels 134. The composite structure 33 and side panels 34 and 134 may comprise two or more separate elements, as shown, or be integrally formed. The illustrated training pant 20 includes a fastening system 80 comprising a cohesive surface 83 on each of the back side panels 134 and a cohesive surface 85 on each of the front side panels 34. When cohesively engaged, the surfaces 83 and 85 form releasable seams 88 that desirably although not necessarily extend substantially the entire distance between the waist opening 50 and the leg openings 52. More specifically, the refastenable seams 88 can cover about 80 to 100 percent, and particularly about 90 to about 98 percent, of the distance between the waist opening 50 and each leg opening 52.

As discussed above, in many instances, the diaper will include a cohesive landing area which cohesively engages with a diaper fastening section, or a fastening region of the diaper itself. This landing area can be formed from a strip of tape comprising a substrate, an adhesive on one side of the substrate to secure the tape to the diaper, and the cohesive surface on the other side of the substrate to engage with the fastener or fastening region. This type of landing area has been used with adhesive fastening systems, with the cohesive surface being replaced with an adhesive-receiving surface. Thus, this type of landing area can be considered a “tried and true” technique in the diaper industry. However, because of the unique characteristics of cohesive surfaces, such as, for example, their non-tackiness, other more efficient methods can be used to create cohesive landing areas.

A cohesive landing area surface can be printed, sprayed, coated, or otherwise applied to the diaper. For example, a cohesive material can be applied to the backsheet material after formation of the diaper, or even before with proper registration procedures, such as at the web-forming stage, or prior to material assembly during the diaper-making process. The applied cohesive can be in any desired configuration or design, such as continuous or discontinuous beads, continuous or discontinuous swirls, meltblown patterns, spray patterns or the like.

Alternatively, a cohesive landing area surface can be integrally formed within the material of the diaper. In some cases, the cohesive material might be such that its presence outside the landing area will not present any real issues and, in fact, might aid in securing a soiled diaper in a wrapped condition for disposal purposes. In other cases, the cohesive material may have to be inactivated, blocked, or voided outside of the landing area, such as by printing or other procedures. Another possibility is to provide the cohesive material in a selectively-activated form (e.g., heat-activated microcapsules) and then activating (e.g., heating) only the landing area.

These landing-cohesive techniques could also be employed with the fastening cohesive surfaces, especially the tabless designs (e.g., FIGS. 4, 5 and 6.) Tab-carried cohesive fastening surfaces might also be formed in these manners and, additionally, by coextrusion with its host substrate. Coextrusion could, of course, also be used to form cohesive landing areas, however this type of technology sometimes does not lend itself to efficient mass-manufacture of garment-like items.

One may now appreciate that the present invention provides diapers, diaper fasteners, and diaper landing areas which incorporate cohesively engaging or engagable surfaces to provide selective closure and/or selective removal of a diaper from a user. While the following description focuses on diapers, it should be clear that the subject invention can be used for any type of absorbent article or garment to be worn by a person for trapping urine or menses. Although the invention has been shown and described with respect to certain preferred embodiments, it is obvious that equivalent and obvious alterations and modifications will occur to others skilled in the art upon the reading and understanding of this specification. The present invention includes all such alterations and modifications and is limited only by the scope of the following claims.

1. A diaper comprising a tapeless cohesive landing area and a cohesive surface for engagement with the tapeless cohesive landing area to secure the diaper in a closed condition, wherein the cohesive landing area comprises a cohesive material printed, sprayed, coated, or otherwise applied to the diaper.

2. A diaper as set forth in claim 1, wherein the cohesive material is printed, sprayed, coated, or otherwise applied to the diaper after its formation.

3. A diaper as set forth in claim 1, wherein the cohesive material is printed, sprayed, coated, or otherwise applied to a web of material used to form the diaper prior to formation of the diaper.

4. A diaper as set forth in any of claim 1, wherein the cohesive landing area comprises a cohesive material integrally formed within the material of the diaper.

5. A diaper as set forth in claim 4, wherein the cohesive material is inactivated, blocked or voided outside of the landing area.

6. A diaper as set forth in claim 4, wherein the cohesive material is activated within the landing area.

7. A diaper comprising a cohesive landing area and a cohesive surface for engagement with the cohesive landing area, the cohesive surface overlaying the backsheet material of the diaper chassis.

8. A diaper comprising a main absorbent body and a strip formed in one piece with at least some sheets of the main absorbent body, said strip having a cohesive surface formed thereon for engagement with a cohesive landing area.

9. A diaper comprising a main absorbent body, ear panels bonded to the main absorbent body, and a strip formed in one piece with each of the ear panels, said strip having a cohesive surface formed thereon for engagement with a cohesive landing area.

10. A diaper as set forth in claim 9, wherein the cohesive surface which engages with the cohesive landing area comprises a cohesive material printed, sprayed, coated, or otherwise applied to the diaper.

11. A diaper comprising a main absorbent body having releasable side seams, the releasable side seams being formed from cohesive surfaces on side edges of the body which cohesively engage to close the seam.

12. A diaper fastening tab comprising a first attachment section for permanent attachment to a first surface of an anchoring area, a landing section for permanent attachment to a landing area, and a fastening section for selective attachment and release from the landing section;
wherein the landing section comprises a cohesive surface and the fastening section comprises a cohesive surface which cohesively bonds therewith for selective attachment and release of the fastening section from the landing section.

13. A diaper fastening tab as set forth in claim 12, wherein the cohesive surface of the landing section and/or the cohesive surface of the fastening section is co-extruded with its host substrate.

14. A diaper fastening tab as set forth in claim 12, wherein the cohesive surface of the landing section and/or the cohesive surface of the fastening section is printed, coated, sprayed or otherwise applied to its host substrate.

15. A diaper fastening tab as set forth in claim 12, wherein the cohesive surface of the landing section and/or the cohesive surface of the fastening section is integrally formed with its host substrate.

16. A diaper fastening tab comprising a first attachment section for permanent attachment to a first surface of an anchoring area and a fastening section for selective attachment and release from a landing area;
wherein the fastening section is elastically joined to the first attachment section; and
wherein the fastening section comprises a cohesive surface which cohesively bonds with a cohesive surface in the landing area.

17. A diaper fastening tab comprising an attachment section for permanent attachment to an anchoring area, a fastening section for selective attachment and release from a landing area, and a central section between the attachment section and the fastening section;
wherein the attachment section, the central section, and the fastening section are folded into a Z-shape when the tab is in a storage condition; and
wherein the fastening section comprises a first portion of a substrate and a cohesive surface on the inner surface of this portion of the substrate for selective cohesive attachment to the landing area.
